(19) United States
(12) Patent Application Publication
(10) Pub. No.: US 2026/0062730 A1
(43) Pub. Date: Mar. 5, 2026

(54) CHIMERIC INVASIN SYSTEM

(71) Applicant: SiVEC Biotechnologies, Inc., Fort Collins, CO (US)

(72) Inventors: Ashley B. Williams, Austin, TX (US); Lyndsey M. Linke, Fort Collins, CO (US); Darcy Mora, Fort Collins, CO (US); Melissa Vivian, Fort Collins, CO (US)

(73) Assignee: SiVEC Biotechnologies, Inc., Fort Collins, CO (US)

(21) Appl. No.: 18/859,453

(22) PCT Filed: Apr. 25, 2023
(86) PCT No.: PCT/US2023/019838

§ 371 Date: Oct. 23, 2024

Related U.S. Application Data
(60) Provisional application No. 63/363,536, filed on Apr. 25, 2022.
(60) Provisional application No. 63/367,518, filed on Jul. 1, 2022.

(51) Int. Cl. C12P 21/02; C07K 14/005; C07K 14/195; C07K 14/37

(52) U.S. Cl. CPC C12P 21/02; C07K 14/005; C07K 14/195; C07K 14/37; C07K 2319/50; C12N 2710/16022; C12N 2710/22022; C12N 2740/16022; C12N 2760/16022; C12N 2770/24122; C12N 2770/32322

(57) ABSTRACT

A transkingdom platform for the delivery of therapeutics to target cells. The system maintains the export and uptake functions of Inv while modifying its targeting away from β1 integrin to other proteins expressed on the surface of target eukaryotic cells (i.e., a cell surface protein) or chemical moieties (i.e., a cell surface chemical moiety) expressed on the surface of a target eukaryotic cell by replacing D4 and D5 of Inv with a binding domain from a heterologous protein via genetic engineering. These heterologous proteins could be derived from bacterial, fungal, animal, or viral genomes. This engineering would result in the construction of a chimeric Inv protein in which D1-D3 (i.e., the non-binding domains) are fused in frame to an alternative binding domain derived from a heterologous protein. The alternative binding domain would interact with a different cell surface protein or chemical moiety, which can in some instances be referred to as a receptor, on the surface on the surface of a eukaryotic cell, thereby allowing specific targeting to cells independent of Inv's intrinsic β1 integrin binding.

### CROSS-REFERENCE TO RELATED APPLICATIONS

This application is the National Stage of International Application No. PCT/US2023/019838, filed Apr. 25, 2023, which application claims the benefit of U.S. Provisional Application No. 63/363,536 filed Apr. 25, 2022 and U.S. Provisional Application No. 63/367,518 filed Jul. 1, 2022.

### REFERENCE TO AN ELECTRONIC SEQUENCE LISTING

The contents of the electronic sequence listing (0111-05-PCT-Chimeric Invasin System.xml; Size: 54,346 bytes; and Date of Creation: Aug. 8, 2023) is herein incorporated by reference in its entirety.

### FIELD OF INVENTION

This invention relates to bacterial delivery vehicles for therapeutic applications. More specifically, this invention relates to bacterial vehicles that can target certain types of eukaryotic cells.

### BACKGROUND OF THE INVENTION

To date, precise in vivo delivery of therapeutic modalities to specifically targeted cells remains challenging. Drug delivery involves two key components: the vehicle itself and the mechanism through with the vehicle specifically arrives at the desired cell type with minimal off-target delivery. Most current delivery strategies are based primarily on mechanical approaches (e.g., electroporation, hydrodynamic injection, and microinjection) and viral vector delivery (e.g., lentivirus, adenovirus, and adeno-associated virus). Non-viral delivery methods, such as liposomes and nanoparticles, are also used, but the size and number of cargo moieties they can carry is extremely limited. While useful in vitro, many of these methods cannot be easily clinically translated to animals or human patients. Bacterial delivery vehicles offer numerous advantages. One particular advantage is their ability to deliver therapeutic moieties intracellularly via invasion. Currently, bacterial delivery vehicles arrive at their targets via passive mechanisms, often dependent on niche-specific biological features (e.g., hypoxic tumor microenvironments). Alternatively, the bacteria are specifically targeted via ligand-receptor interactions with factors on the target cell surface. To date, even the most specific targeting method can have off-target effects to expression of the targeting ligand across multiple cell types in multiple tissues and organs. To realize the full potential of bacterial delivery vehicles, more specific targeting mechanisms are required to mitigate off-target effects (i.e., delivery to unwanted or undesirable tissue and cell types).

### SUMMARY OF THE INVENTION

The present invention provides systems and methods for the specific targeting to cells in a eukaryotic host employing highly specific targeting of an invasive, non-pathogenic bacterial delivery vehicle where the bacterial delivery vehicle has been engineered to produce a chimeric invasin (Inv) polypeptide having a modified binding domain. The Inv polypeptide in its non-chimeric form has five domains referred to as D1, D2, D3, D4, and D5, along with a beta-barrel which traverses the outer membrane of the bacterium.

In certain aspects, the invention provides systems and methods to maintain the export (i.e., export to the surface of the bacterial cell) and uptake functions of the Inv protein from Yersinia pseudotuberculosis, while modifying its targeting from β1 integrin to other protein domains expressed on the surface of target eukaryotic cells (i.e., a cell surface protein) or chemical moieties (i.e., a cell surface chemical moiety) expressed on the surface of a target eukaryotic cell by replacing D4 and D5 of Inv with a binding domain from a heterologous protein via genetic engineering. These heterologous proteins could be derived from bacterial, fungal, animal, or viral genomes. This engineering would result in the construction of a chimeric Inv protein in which D1 through D3 (i.e., the non-binding domains) from Inv are fused in frame to an alternative binding domain derived from a heterologous protein or a synthetic binding domain. The alternative binding domain would interact with a different cell surface protein or chemical moiety, which can in some instances be referred to as a receptor, on the surface of a eukaryotic cell, thereby allowing specific targeting to cells independent of Inv's intrinsic β1 integrin binding. In some instances, the heterologous protein's binding domain can be referred to as a ligand-binding domain.

It is contemplated that one could take a non-binding domain from Inv (e.g., D1, D2 and D3) or full-length Inv, add a linker sequence such as those described immediately below, and then add a binding domain from one of the proteins listed in Tables 1, 2, or 3. Thus, one could utilize a sequence from Inv such as that provided below, SEQ ID NO. 1, from about amino acid 1 to up to about amino acid 795 or from about amino acid 1 to about amino acid 986, or something that is 95% or 90% identical thereto.

In certain aspects, the present invention provides a nonpathogenic bacterium that has been engineered to express a chimeric targeting ligand. The bacterium is engineered to have a sequence encoding the non-binding domains of an Inv protein (e.g., D1, D2 and/or D3) fused to a sequence encoding a heterologous binding domain (see e.g., Tables 1-3 below for proteins where the binding site from the protein can be utilized). Thus, a chimeric Inv protein having an altered binding domain upon expression of the sequence can be generated. This can allow the transkingdom delivery vehicle to be targeted to other tissues than can be achieved when using a binding domain targeting the β1 integrin. SEQ ID. NO. 1, below, discloses an amino acid sequence for an Inv polypeptide. It is contemplated that the D1, D2 and/or D3 regions of that sequence could be used to construct a chimeric protein, such as by conversion to the corresponding nucleic acid sequence or a sequence having 99%, 95% or 90% homology to a nucleic acid sequence of the D1, D2 and/or D3 region (See SEQ ID NO. 38). Because D1-D3 facilitate binding to target cells but not invasion, bacteria expressing a chimeric targeting ligand comprising D1-D3 fused to a heterologous binding domain targeting a specific factor on the surface of the target cells could be used for cell labeling or detection.

In further embodiments, the present invention provides a nonpathogenic bacterium that has been engineered to express a chimeric targeting ligand where the bacterium is engineered to have a sequence encoding the complete Inv protein (i.e., D1, D2, D3, D4, D5) fused to a sequence encoding the binding domain from a heterologous protein or a synthetic binding domain (see e.g., Tables 1-3 below for proteins where the binding site from the protein can be utilized). Thus, a chimeric Inv protein having an altered binding domain upon expression of the sequence can be generated. This can allow the transkingdom delivery vehicle to be targeted to other cell types and tissues than can be achieved using a binding domain targeting β1 integrin. SEQ ID. No. 1, below, discloses an amino acid sequence for an Inv polypeptide. It is contemplated that the D1, D2, D3, D4, and D5 regions of that sequence could be used to construct a chimeric protein, such as by conversion to the corresponding nucleic acid sequence or a sequence having 95% or 90% homology to a nucleic acid sequence encoding the D1, D2, D3, D4, and D5 regions of Inv.

In the construction of fusion (i.e., chimeric) proteins, various linker sequences, a specific sequence of amino acids, can be used to connect the protein domains (i.e., independently folding amino acid sequences). Linker sequences are normally categorized as rigid or flexible linkers, and some might contain a cleavage site as described herein. The physical features of linkers can influence key properties of fusion proteins, including expression level, biological activity, or other in vivo behaviors. Linker sequences containing glycine and serine are generally flexible, allowing the domains to move independently relative to one another. Linker sequences containing proline tend to be more rigid, limiting the relative motion of the domains [see generally Chen X, Zaro J L, Shen W C. Fusion protein linkers: property, design and functionality. Adv Drug Deliv Rev. 2013 October;651(10):1357-69. doi: 10.1016/j.addr.2012.09.039. Epub 2012 Sep. 29. PMID: 23026637; PMCID: PMC3726540.]

In further aspects, chimeric polypeptides according to the invention utilize a sequence that includes a linker sequence to link the non-binding domains of Inv fused to the sequence encoding a heterologous binding domain. The linker sequence can be a sequence selected from SEQ ID NOS. 2-20, as disclosed below. In certain embodiments the binding domain sequence is a sequence encoding a binding domain selected from any one of the polypeptides referred to in Tables 1-3.

In an advantageous embodiment the chimeric targeting ligand utilizes a sequence that includes a composite linker sequence to link the non-binding domains of Inv or full-length Inv to the sequence encoding the heterologous binding domain (BD), including a synthetic binding domain. The first part (N-terminal end) of the linker sequence can be a sequence selected from SEQ ID NOS. 2-20, as disclosed below. The second part of the linker can comprise a cleavage site or cleavage sites for one or more of the peptidases or proteases provided in Table 4. Use of a composite linker serves to confirm that the correct target cell type has been reached due to the required presence of the peptidase(s) or protease(s) on the target cell surface to allow invasion via removal of the heterologous BD. In certain embodiments the BD sequence is a sequence encoding a BD selected from any one of the polypeptides referred to in Tables 1-3 or a synthetic BD.

The nonpathogenic bacterium engineered to express a chimeric Inv polypeptide according to the various aspects can utilize a sequence encoding the non-binding domains of an Inv protein that encodes a polypeptide that is 90% (or 95% or even 99%) identical to amino acids 1-794 of SEQ ID NO. 1, presented below.

The nonpathogenic bacterium engineered to express a chimeric targeting ligand can alternatively utilize a sequence encoding all domains of an Inv protein that is 90% (or 95% or 99%) identical to amino acids 1-986 of SEQ ID NO. 1.

The nonpathogenic bacterium engineered to express a chimeric Inv polypeptide can be further engineered to express therapeutic nucleic acids, proteins, antibodies, antibody derivatives, polypeptides, gene-editing systems (CRISPR and other gene editing nucleases), eukaryote-translatable mRNA or combinations thereof. The therapeutic nucleic acids, proteins, antibodies, antibody derivatives, polypeptides, gene-editing systems (CRISPR and other gene editing nucleases), eukaryote-translatable mRNA or combinations thereof can be expressed from a sequence on the chromosome of the bacterium or on a plasmid.

In an further aspect, the present invention provides a nonpathogenic bacterium engineered to express a chimeric Inv polypeptide. The expressed chimeric Inv polypeptide can have non-binding domains (e.g., D1, D2 and/or D3) of an Inv protein or the full-length Inv (D1-D5) fused to a heterologous binding domain to generate a chimeric Inv protein. A chimeric targeting ligand produced by the bacterium can be used to alter the type of target cell or tissue for the bacterial delivery vehicle.

In still further aspects, the present invention provides a bacterium for nucleic acid delivery, or delivery of another molecule (e.g, proteins, antibodies, antibody derivatives, polypeptides, gene-editing systems (CRISPR and other gene editing nucleases), eukaryote-translatable mRNA) to a eukaryotic cell comprising a nonpathogenic bacterium, where the bacterium has been engineered to express at least one invasion factor and where the invasion factor has the non-binding domains of an Inv protein or full-length Inv fused to a heterologous binding domain to generate a chimeric Inv protein. The heterologous protein can be a protein that binds to a cell surface protein or cell surface chemical moiety on a target eukaryotic cell. The binding domain can be a fragment of the heterologous protein, such as one created by not including non-binding regions of the heterologous protein. In an advantageous embodiment the binding domain of the heterologous protein is translated from a sequence that is encoded in a bacterial, fungal, viral, or animal genome, but engineered to be encoded and translated as a part of a chimeric Inv protein by the bacterial delivery vehicle. Alternatively, the binding domain can be any synthetic (i.e., non-natural) protein that facilitates binding to a target cell surface.

The chimeric Inv targeting ligand can be expressed from a sequence on the chromosome of the engineered bacterial delivery vehicle or a plasmid carried by the engineered bacterial delivery vehicle.

In an advantageous embodiment, the chimeric Inv targeting ligand has a peptide linker that is fused between the non-binding domains of an Inv protein and a binding domain from a heterologous protein. The peptide linker can have one or more amino acids fused in-frame to the non-binding domains of an Inv protein and the binding domain from a heterologous protein. The non-binding domains of the Inv protein can be the D1, D2, and D3 domains of Inv or a combination or subset thereof or full-length Inv.

In an advantageous embodiment, the chimeric Inv protein has a peptide linker that is fused between the non-binding domains of an Inv protein and a binding domain from a heterologous protein. The peptide linker can have one or more amino acids fused in-frame to the full-length Inv protein and a heterologous binding domain.

The bacterium for nucleic acid delivery to a eukaryotic cell can be further engineered to express a therapeutic nucleic acid from a sequence on the chromosome of the bacterium or from a plasmid.

Accordingly, in certain aspects the present invention provides an expression cassette for the production of a chimeric invasin (Inv) polypeptide. The expression cassette can include a prokaryotic promoter and a nucleic acid sequence encoding an Inv polypeptide fused to a linker polypeptide at the carboxy terminus of the Inv polypeptide. Expression of the nucleic acid encoding the chimeric invasin polypeptide is controlled by the prokaryotic promoter. In certain advantageous embodiments the expression cassette for the production of a chimeric invasin polypeptide includes a sequence encoding a binding domain to bind a surface moiety on a target cell. The binding domain is fused to the amine terminus of the linker polypeptide. The binding domain can be a binding domain from a protein listed in Tables 1-3 or the binding domain can a synthetic binding domain. The nucleic acid sequence of the invasin region of the chimeric polypeptide can be 90% identical, 95% identical or even 99% identical to nucleic acids 1-2958 of SEQ ID. NO. 37. Similarly, the nucleic acid sequence of the invasin region of the chimeric polypeptide is 90% identical, 95% identical or even 99% identical to nucleic acids 1-2382 of SEQ ID. NO. 37 or SEQ ID. NO. 38.

The expression cassette for the production of a chimeric invasin polypeptide according to the first aspect can further include a nucleic acid sequence encoding a linker polypeptide and/or a protease cleavage site. Exemplary linkers include SEQ. ID. NO. 2 through SEQ ID. NO. 20. Exemplary protease cleavage sites include SEQ. ID. NO. 22 through SEQ ID. NO. 36

The sequence encoding the protease cleavage site cleaved by a peptidase or protease can be located between the sequence encoding the invasin and the sequence encoding the binding domain.

The expression cassette for the production of a chimeric invasin polypeptide can use a prokaryotic promoter such as the T7, lacUV5, gapA, T5, recA, Ptac, Patac, pAl, lac, Sp6, araBad, and trp promoters. The prokaryotic promoter could also be a hybrid or synthetic prokaryotic promoter.

A bacterium expressing a chimeric invasin polypeptide can be constructed where the bacterium is engineered to include the expression cassette for the production of a chimeric invasin polypeptide. The bacterium can be a bacterium selected from the group consisting of Clostridium difficile, Escherichia coli, Clostridium tetani, Helicobacter pylori, Fusobacterium micleatum, Gardnerella vaginitis, Porphyromonas gingivalis, Aggregatibacter actinomycetemcomitans, Listeria monocytogenes, Staphylococcus aureus, Campylobacter jejuni, Vibrio vulnificus, Salmonella typhi, Clostridium botulinum, Mycobacterium tuberculosis, Mycobacterium leprae, Mycobacterium lepromatosis, Corynebacterium diptheriae, Klebsiella pneumoniae, Acinetobacter baumannii, Streptococcus mutans, group B streptococci, Staphylococcus aureus, Streptococcus agalactiae, Streptococcus pneumonia, Enterococcus spp., Enterococcus faecalis, Listeria, Yersinia, Rickettsia, Shigella, Salmonella spp., Legionella, Chlamydia, Brucella, Neisseria, Burkolderia, Bordetella, Borrelia, Coxiella, Mycobacterium, Helicobacter, Staphylococcus, Streptococcus, Porphyromonas, Vibrio, Treponema, Lactobacillus, and Bifidobacteriae. In an advantageous embodiment the bacterium expressing a chimeric invasin polypeptide is an Escherichia coli bacterium.

In still further aspects the present invention provides method for treating or preventing a disease in a subject by administering a bacterium expressing a chimeric invasin polypeptide where bacterium is further engineered to express a therapeutic nucleic acid produced by the bacterium. Similarly, in addition to expressing a chimeric invasin polypeptide, the bacterium can be engineered to express therapeutic nucleic acids, proteins, antibodies, antibody derivatives, polypeptides, gene-editing systems (CRISPR and other gene editing nucleases), eukaryote-translatable mRNA or combinations thereof from a sequence on the chromosome of the bacterium or on a plasmid.

The present invention also provides a chimeric invasin polypeptide. The chimeric invasin polypeptide can include an Inv polypeptide and a linker polypeptide, wherein the linker polypeptide has a first end (N-terminus) and a second end (C-terminus), wherein the first end (N-terminus) of the linker polypeptide is attached to the C-terminus of the Inv polypeptide. The chimeric invasin polypeptide can further include peptidase or protease cleavage site.

In an advantageous embodiment the chimeric invasin polypeptide further includes a binding domain of a heterologous protein or a synthetic binding domain attached to the second end (C-terminus) of the linker polypeptide. Advantageous binding domains can be a binding domain from a protein listed in Tables 1-3.

The Inv polypeptide amino acid sequence of the chimeric invasin can have a sequence that is 90% (or 95% or 99%) identical to amino acids 1-986 of SEQ ID. NO. 1 or 90% (or 95% or 99%) identical to amino acids 1-985 of SEQ ID. NO. 39.

The Inv polypeptide amino acid sequence of the chimeric invasin can have a sequence that is 90% (or 95% or 99%) identical to amino acids 1-794 of SEQ ID. NO. 1 or 90% (or 95% or 99%) identical to amino acids 1-794 of SEQ ID. NO. 39.

In still further aspects the present invention provides a composition for the selective binding of a substrate to a target molecule. The composition can utilize a chimeric invasin polypeptide conjugated at the amino terminal of the Inv polypeptide to a biologic and synthetic substrate surface, wherein the substrate is selected from the group consisting of beads, viruses, exosomes, rigid substrates (e.g., for production of a lateral flow strip), paper-based biosensors, plastic substrates (e.g., for production of a plastic-based biosensor), graphene-based substrates, or nanomaterials (e.g., a lipid nanoparticle, metallic nanoparticles, mesoporous silica nanoparticles, nanowire, ITO, organic polymers).

In still further aspects the present invention provides a chimeric invasin polypeptide comprising the D1-D3 domains of the Inv polypeptide attached to the binding domain of a heterologous protein or a synthetic binding domain. The chimeric invasin polypeptide can include a linker, wherein the linker polypeptide has a first end (N-terminal) and a second end (C-terminal), where the first end of the linker polypeptide is attached to the C-terminal amino acid of the Inv polypeptide and the second end of the linker polypeptide is attached to the amino-terminal of the binding domain of the heterologous protein or the amino- terminal of the synthetic binding protein.

### BRIEF DESCRIPTION OF THE DRAWINGS

For a fuller understanding of the invention, reference should be made to the following detailed description, taken in connection with the accompanying drawing, in which:

FIG. 1 is a series of four illustrations (labeled (A)-(D)) of the structure of full-length Inv (A) and the structures of various versions of chimeric Inv proteins described herein ((B)-(D)). The chimeric Inv proteins comprise D1-D3 of Inv fused to the binding domain of a heterologous protein (FIG. 2B) or full-length Inv fused to a linker sequence that may or may not contain a peptidase or protease cleavage site (FIG. 2C and FIG. 2D).

FIG. 2 is a drawing the shows the three-step chimeric ligand targeting and invasion paradigm described herein.

FIG. 3 is a micrograph of A549 cells treated with FEC19 bacteria harboring pSi_1fHER2-scr.c. The image (A549 cells) confirms that the bacterial delivery vehicle can be specifically targeted to and then invade HER2-positive, furin-positive A549 cells while it cannot invade HER2-negative HeLa cells.

FIG. 4 is a drawing (A) and a schematic (B) that shows the two-factor chimeric Inv protein used in the Example below. FIG. 4A shows the schematic of the chimeric Inv protein, which comprises D1-D5 of Inv linked in frame to a synthetic nanobody that binds to HER2. The linker comprises the cleavage site for the cell-surface protease furin. FIG. 4B provides a schematic of the sequence of the chimeric Inv protein illustrated in (A). Only the C-terminal end of Inv D5 is shown for illustrative purposes.

FIG. 5 is a pair of diagrams labeled (a) and (b) providing an annotated linear representation of the Inv amino acid sequence, with the amino acids comprising D1-D4/D5 labelled with the domain name and function. The chimeric Inv protein described herein would comprise D1, D2, and D3, and D4/D5 would be replaced with a heterologous binding domain. The diagrams in (a) and (b) are identical, with (b) rotating the diagram in (a) by 90 degrees to enlarge the data presented therein. The sequences shown in (A) and (B) are identical and presented as SEQ. ID. NO. 1, below.

### DETAILED DESCRIPTION OF THE PREFERRED EMBODIMENT

The present invention provides a system for the targeted intracellular delivery of therapeutic or non-therapeutic moieties to eukaryotic cells using a non-pathogenic bacterial delivery platform expressing a bifunctional chimeric targeting-invasion factor (“chimeric targeting ligand”) that interacts with and binds to a factor (“receptor”) on the surface of the target cell and then triggers internalization of the bacterial delivery vehicle by the target cell. The chimeric ligand contains a constant region, comprising the Yersinia pseudotuberculosis invasin (Inv) protein (encoded by the inv gene), and a variable region that is customized for the targeting purpose. The variable region comprises a peptide or protein that binds to the receptor on the target cell. The variable region could, for example, comprise a single-domain antibody, a nanobody, a camelid IgG antibody, a llama IgG antibody, peptibodies, any other immune polypeptide, or any peptide comprised of amino acid residues that bind specifically with a receptor molecule found on the outer membrane of a eukaryotic cell. This chimeric ligand could target the bacteria to a specific cell type or to a class of cell types expressing the same receptor.

The Gram-negative genus of Yersinia comprises at least seventeen species, of which three are human and animal pathogens: Y. enterocolitica, Y. pseudotuberculosis, and Y. pestis.

The pathogenicity of these bacteria depends on factors that allow them to adhere to cells and cross the cell membrane to reach the target cell cytoplasm. These organisms express a variety of such factors, including invasin (Inv), YadA, YadB, YadC, Ail, Pla, and Ph 6 antigen. These various proteins are together known as adhesins and each protein acts at specific stages of the host-pathogen interaction. Importantly, each of these proteins bind a range of host factors, including b1 integrins, collagen, fibronectin, laminin, and complement-related factors. Furthermore, all of these proteins are anchored to the outer membrane (OM) of the bacteria where they form rod-like structures. Presentation on the OM allows the proteins to mediate interactions with factors on the surface of their target cells. The transport from the cytoplasm of the bacterial cell to the OM can also occur via various mechanisms. It is contemplated that a YadA, YadB, YadC, Ail, Pla, and Ph 6 antigen could be used to construct a chimeric polypeptide by replacing the inv nucleic acid sequence for in the expression for a sequence encoding YadA, YadB, YadC, Ail, Pla, and Ph 6 antigen, which could create an alternative chimeric targeting bacterium. So, for example, a YadA chimeric polypeptide could include a binding domain such as taught herein for a chimeric invasin, and further optionally including a linker sequence and/or a cleavage site between the YadA amino acids and the BD amino acids. Sequences for these adhesins are known such as for YadA (UniProt P31489-YADA1_YEREN; UniProt P10858-YADA_YERPS, which are incorporated by reference). A sequence could be utilized for the respective adhesin that is 90% identical, 95% identical or 99% identical to the consensus sequence for YadA, YadB, YadC, Ail, Pla, or Ph 6 antigen.

Invasin is the first adhesin expressed during invasion by enteropathogenic Yersinia species (spp.). Its primary role is the invasion of epithelial cells via β1 integrin binding, which allows the bacterium to initiate colonization and internalization of host epithelial cells. Invasin has a modular structure comprising several clearly defined functional sequences. Most broadly, Inv contains the structural elements associated with autotransporters: a beta-barrel “transporter” structure at the amino (N)-terminus and an extracellular “passenger” domain at the carboxy (C)-terminus. The passenger domain autonomously passes from the periplasm to the outer membrane (OM) without the need for energy sources (e.g., ATP). In the case of Inv, this transport is thought to be mediated by passage of the protein into the periplasm via an N-terminal signal peptide followed by insertion of the beta-barrel domain into the OM to form a pore for the passenger domain to pass through. The structure of the passenger domain is highly modular, contains five protein domains (D1-D5). The secondary structure of D1-D4 comprises mostly beta sheets, while that of D5 comprises an alpha helix/beta helix secondary structure. Together, D4 and D5 form a module that binds to integrins with high affinity.

In certain aspects, the present invention provides a bacteria-mediated delivery vehicle that comprises invasive, non-pathogenic bacteria that express and then export the chimeric ligand to the outer membrane of the bacterial cell. The bacteria can contain a prokaryotic expression cassette encoding the chimeric ligand under the control of a prokaryotic promoter (synthetic or endogenous). The novel bacterial delivery platform expressing and presenting this ligand can provide cell-specific and tissue-specific delivery and intracellularization of the delivery vehicle in any eukaryotic cell in any cell cycle stage (dividing, non-dividing, quiescent) as long as the cell expresses the cognate cell surface receptor. Targeting to desired eukaryotic cells can be controlled via the selection of a variable region that is specific to a receptor on the target eukaryotic cell.

In further aspects this invention advances the delivery of therapeutic nucleic acids, proteins, antibodies, antibody derivatives, polypeptides, gene-editing systems (CRISPR and other gene editing nucleases), and eukaryote-translatable mRNA using an E. coli transkingdom delivery vehicle by allowing precise targeting of the bacteria to target eukaryotic cells expressing specific surface proteins or chemical moieties. Delivery of therapeutic modalities are discussed such as in U.S. Pat. No. 11,312,954 B2 to Linke et al. and US 2022/0364122 A1 to Linke et al., the contents of which are incorporated by reference. The transkingdom bacterial delivery vehicle must target and invade specific cell types for intracellular cargo delivery; however, the targeting and the invasion are not trivial or passive processes, especially when the target cell does not naturally take up the bacteria via, for example, phagocytosis.

Bacteria use various invasion factors to invade non-phagocytic cells as exemplified by Yersinia pseudotuberculosis (Mikula et al., 2012). These bacteria depend on a surface-presented invasion factor protein, invasin protein (Inv), that binds to β1 integrin on the surface of target eukaryote cells. Following binding, intrinsic properties of Inv stimulate uptake of the bacteria by the otherwise non-phagocytic eukaryotic cell. This uptake process depends on three specific properties of Inv: 1) export of Inv to the bacterial surface, 2) binding of Inv to β1 integrin on the cell surface, and 3) stimulation of bacterial uptake.

The Y. pseudotuberculosis Inv protein is a multi-domain protein, comprising five independently folding domains, D1, D2, D3, D4, and D5. The primary accession number for the Inv protein is UNIPROT P11922 and the inv gene is YPTB1668 (Isberg et al., 1987, Leong et al., 1990, Chain et al., 2004), full sequence of which is present in Table 5, below. The critical invasive functions of Inv mentioned above are compartmentalized into these various domains. D1, D2, and D3 are responsible for Inv export to the bacterial surface and stimulation of cellular uptake, while D4 and D5 are required for β1 integrin binding (FIGS. 7 and 8) (Dersch and Iseberg, 2000). Inv is an autotransporter protein (Leo et al., 2014), meaning that its export to the bacterial cell surface is an intrinsic property of the protein, i.e., it does not require any separate export mechanism. Therefore, by separating the domains of Inv, so can its functions of export, uptake stimulation, and targeting be separated and leveraged independently for targeting and invasion of the transkingdom delivery vehicle.

This invention describes an approach to maintain the export and uptake functions of Inv while modifying its targeting away from β1 integrin to other proteins expressed on the surface of target eukaryotic cells (i.e., a cell surface protein) or chemical moieties (i.e., a cell surface chemical moiety) expressed on the surface of a target eukaryotic cell by replacing D4 and D5 of Inv with a binding domain from a heterologous protein or a synthetic (i.e., non-natural) binding domain or by fusing full-length Inv to a binding domain from a heterologous protein or a synthetic (i.e., non-natural) binding domain via genetic engineering. The heterologous proteins could be derived from bacterial, fungal, animal, or viral genomes. Alternatively, the BD could comprise a synthetic protein (i.e., a protein that does not occur naturally). The source of the synthetic BD could be laboratory procedures generally based on biochemical approaches or computational discovery (e.g., via computer modeling or artificial intelligence). The synthetic BD could be a single-domain antibody, a nanobody, or any other ligand that binds to a moiety on the surface of target cells. This engineering would result in the construction of a chimeric Inv protein in which D1-D3 (i.e., the non-binding domains) are fused in frame to an alternative heterologous binding domain or a chimeric Inv protein in which Inv D1-D5 (i.e., full-length Inv) are fused in frame to a heterologous binding domain. The alternative binding domain would interact with a different cell surface protein or chemical moiety than the intrinsic binding domain of Inv, which can in some instances be referred to as a receptor, on the surface on the surface of a eukaryotic cell, thereby allowing specific targeting to cells independent of Inv's intrinsic β1 integrin binding. In some instances, the heterologous protein's binding domain can be referred to as a ligand-binding domain. Examples of bacterial heterologous proteins and their binding partners (protein or chemical) are given in Table 1. Examples of fungal heterologous proteins and their binding partners (protein or chemical) are given in Table 2. Examples of viral heterologous proteins and their binding partners (protein or chemical) are given in Table 3. Examples of animal heterologous proteins that contain binding domains include glycan binding proteins and cell adhesion proteins (e.g., GalNAc binding proteins, lectins, the group of cell adhesion molecules (CAMs), the group of sulfated glycosaminoglycan (GAG)-binding proteins, selectins, integrins, laminin, cadherins, fibronectin, collagens, thrombospondin, vitronectin, tenascin, apolipoproteins B, E, and A-V, lipoprotein lipase, hepatic lipase, Siglecs, galectins, immunoglobulins, and annexins, among others).

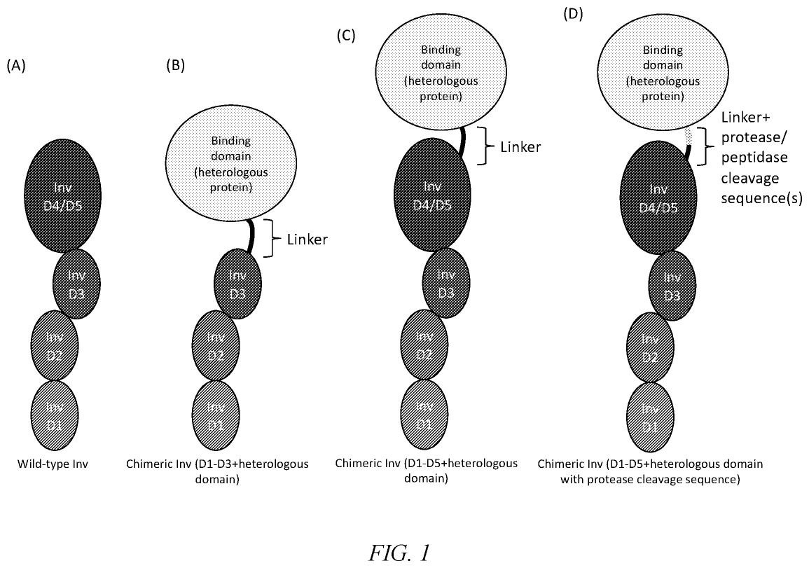

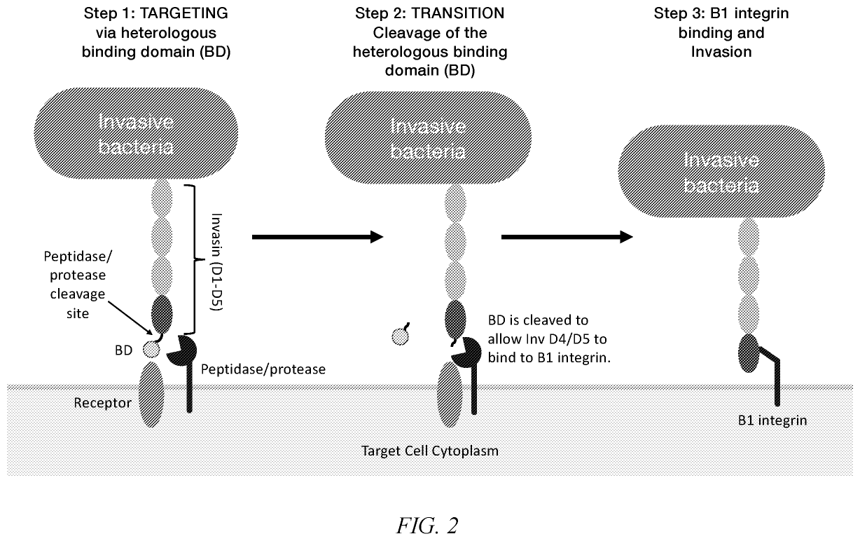

Invasive factors (e.g., the SARS-CoV2 virus) interact with and invade their target cells via a multi-step process in which the invasive factor first binds to a receptor on the target cell surface via a specific binding moiety followed by proteolytic processing of the binding moiety to enable or enhance invasion. This proteolytic processing occurs when a protease or peptidase cleaves the protein at a specific cognate cleavage site (e.g., the SARS-CoV2 spike protein must be cleaved at a furin cleavage site). This strategy can help optimize the functions of ligand binding and invasion. [See e.g., Jackson, C. B., Farzan, M., Chen, B. et al. Mechanisms of SARS-CoV-2 entry into cells.Nat Rev Mol Cell Biol 23, 3-20 (2022); see also Pager C T, Dutch R E. Cathepsin L is involved in proteolytic processing of the Hendra virus fusion protein. J Virol. 2005 October; 79(20):12714-20. doi: 10.1128/JVI.79.20.12714-12720.2005. PMID: 16188974; PMCID: PMC1235853; Carruthers V B, Blackman M J. A new release on life: emerging concepts in proteolysis and parasite invasion. Mol Microbiol. 2005 March;55(6):1617-30. doi: 10.1111/j 1365-2958.2005.04483.x. PMID: 15752188.]. Via genetic engineering, a similar paradigm can be applied to the bacterial delivery platform described herein. To construct such a bifunctional invasion system, a site recognized and cleaved by a peptidase or protease (including, but not limited to those of the proteins given in Table 4, below) is placed in frame between the Inv sequence and the heterologous binding domain sequence (see FIG. 1). The bacterial delivery vehicle then enters the target cells via a three-step process (FIG. 2): (1) Targeting: the heterologous binding domain recognizes and binds to a receptor on the target cell surface, thereby targeting the bacterial vehicle to a specific cell type. (2) Transition: the heterologous binding domain is cleaved from the chimeric Inv protein by a specific peptidase or protease found on the target cell surface to activate the invasive function of the Inv protein. (3) Invasion: the activated Inv protein binds to β1 integrin on the target cell surface to facilitate invasion of the target cell.

The function of multi-domain proteins such as Inv require specific topological interactions between their own domains or with other binding partners (e.g., proteins or chemical moieties). One critical feature of a protein that can influence these topological interactions is the spacing between its internal domains as determined by a specific amino acid sequence (i.e., a linker peptide) (Chen et al., 2012). In the case of chimeric Inv this is the spacing between the non-binding domain and the binding domain; therefore, when engineering a chimeric Inv protein it might be advantageous to modify the amino acid sequence of the linker peptide between the domains to modulate these interactions to optimize binding of the chimeric Inv protein to its binding partner on the surface of the eukaryotic cell. This modification can be made by altering the amino acid sequence of inter-domain linker peptides (i.e., peptide linkers) to modulate flexibility and spacing. Examples of peptide linker amino acid sequences that could be useful include [SEQ. ID. NO. 2] EAAAREAAAR, [SEQ. ID. NO. 3] EAAAREAAAREAAAREAAAR, [SEQ. ID. NO. 4] GSGSGS, [SEQ. ID. NO. 5] GSGSGSGSGS, [SEQ. ID. NO. 6] GGGS, [SEQ. ID. NO. 7] GGGGS, [SEQ. ID. NO. 8] GGGSGGGGSGGGS, [SEQ. ID. NO. 9] GGSG, [SEQ. ID. NO. 10] GGSGGGSG, [SEQ. ID. NO. 11] GGSGGGSGGGSG, [SEQ. ID. NO. 12] GSGGS, [SEQ. ID. NO. 13] GSSGS, [SEQ. ID. NO. 14] ACGSLSCGSF, [SEQ. ID. NO. 15] GENLYFQSGG, [SEQ. ID. NO. 16] SACYCELS, [SEQ. ID. NO. 17] RPACKIPNDLKQKVMNH, [SEQ. ID. NO. 18] PPPYQPLGGGGS, [SEQ. ID. NO. 19] WRKRLRKKRLRKKRRLKKRRRKKQRRKRR, LEGSGQGPGSGQGSGSPGSGQG and [SEQ. ID. NO. 20] GS. It is contemplated that one could take a non-binding domain from Inv (e.g., D1, D2 and D3) or full-length Inv, add a linker sequence such as those described immediately above, and then add a binding domain from one of the proteins listed in Tables 1, 2, or 3 or a synthetic binding protein/binding domain. Thus, one could utilize a sequence such as that provided in FIG. 1 from about amino acid 1 to up to about amino acid 795 or from about amino acid 1 to about amino acid 986, or something that is 95% or 90% identical thereto.

This invention advances the delivery of nucleic acids, proteins, antibodies, antibody derivatives, polypeptides, gene-editing systems, and eukaryote-translatable mRNA by providing a bacterial delivery platform that can be further tailored to target specific cell surface proteins and cell surface chemical moieties for more precise nucleic acid, protein, antibody, antibody derivative, polypeptides, gene-editing systems, and eukaryote-translatable mRNA delivery. The described export and uptake domains of Inv fused (or linked) with the binding domain of a heterologous or synthetic protein can be encoded in the bacterial cell via genomic or plasmid expression. Similarly, it is often advantageous to express the nucleic acid-encoding sequences from the bacterial chromosome rather than from a plasmid for multiple reasons, including low metabolic burden to the host cell, expression level stability, genetic stability, and no requirement for a selective agent (Ou, et al., 2018).

It is also contemplated that the described export and uptake domain of Inv or full-length Inv fused with the binding domain of a heterologous protein (chimeric Inv) could be expressed, added, or conjugated to other biologic and synthetic surfaces. These include beads, viruses, exosomes, rigid substrates (e.g., for production of a lateral flow strip), paper-based biosensors, plastic substrates (e.g., for production of a plastic-based biosensor), graphene- based substrates, or nanomaterials (e.g., a lipid nanoparticle, metallic nanoparticles, mesoporous silica nanoparticles, nanowire, ITO, organic polymers).

Exosomes, liposomes and other lipid vesicles have been used as nucleic acid delivery platforms to carry RNA payloads for delivery to distant tissues. Delivery vehicles such as liposomes have drawbacks including leakage of vesicle content, batch-to-batch variation, high cost of production, and limited targeting ability. This transkingdom delivery system is based on the use of a non-pathogenic bacterial-mediated RNAi delivery vehicle that uses receptor-mediated phagocytosis for specific intracellular delivery at the tissue site of action, resulting in the accumulation of shRNAs in endosomes and the efficient release of the shRNA payload into the target cell's cytoplasm for RNAi silencing. These transkingdom vehicles have been Escherichia coli (E. coli) cells that have been engineered to specifically target mucosal epithelial tissues and deliver a payload of constitutively generated shRNAs in a sequence-independent manner.

Example. Bacterial vehicle invasion of HER2-positive cancer cells via the chimeric Inv targeting ligand (2-factor invasion paradigm).

Successful invasion of HER2-positive cancer cells by a chimeric Inv targeting ligand comprising D1-D5 of Inv, a linker containing a furin cleavage site, and a nanobody specific for HER2, a cell-surface presented protein on the target cells, was demonstrated via an invasion assay and laser scanning confocal microscopy.

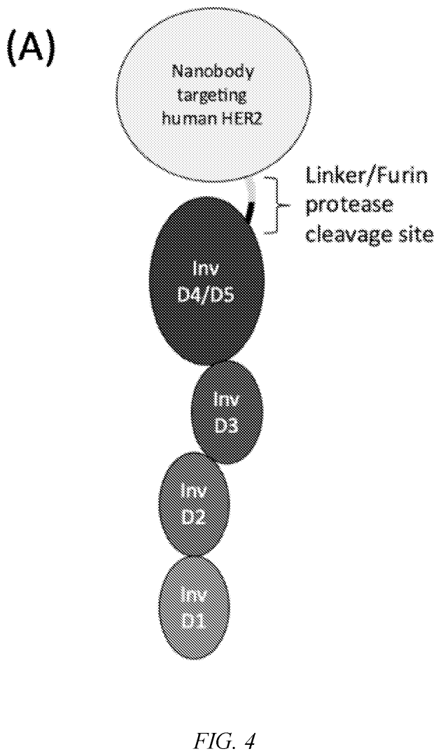

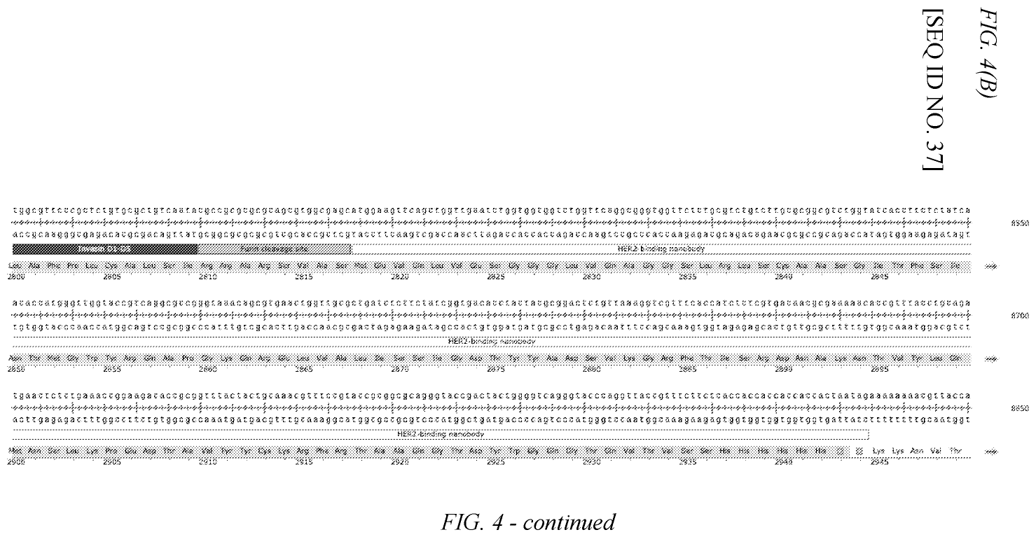

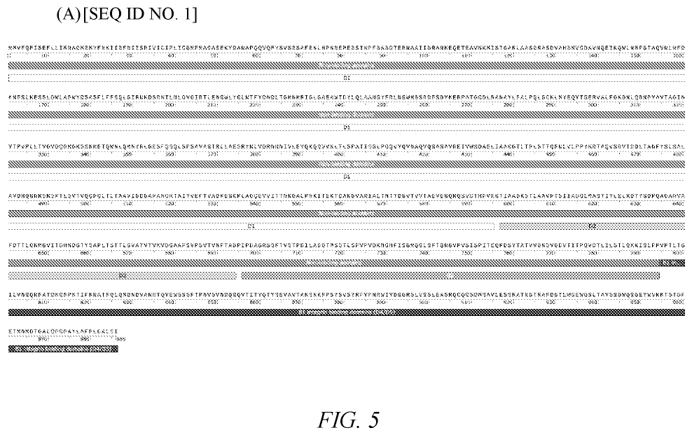

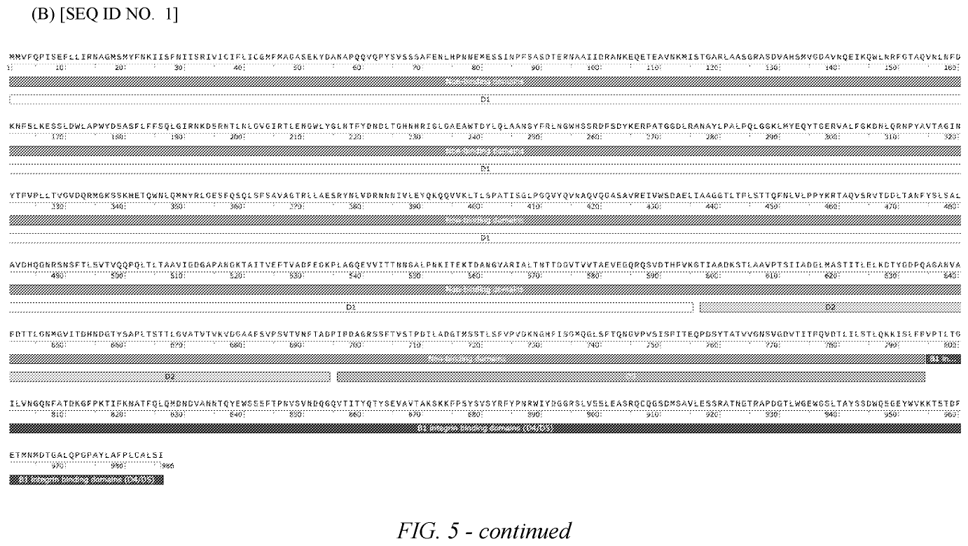

A plasmid (“pSi_1fHER2-scr.c”) encoding a chimeric Inv protein comprising D1-D5 of Inv linked to a synthetic (i.e., non-natural) nanobody via an in-frame furin protease cleavage site was constructed via molecular cloning as in FIG. 4. In this example, the linker is a compound linker, i.e., a generic linker sequence with a fused furin protease cleavage site. The nanobody binds specifically to HER2, a receptor expressed on the surface of the eukaryotic target cells. Bacterial transcription of the chimeric Inv protein is constitutive under the control of a modified lac (15 promoter and transcription is terminated via a standard bacterial transcriptional terminator. After transcription-translation of the chimeric Inv protein by the bacteria, the protein is translocated to the surface of the bacteria via the auto-export activity of domains D1-D3 of Inv. pSi_1fHER2-scr.c was transformed into E. coli bacteria (FEC19), which are non-invasive in the absence of the chimeric Inv D1-D5, which are included on pSi_1fHER2-scr.c plasmid. Transformed FEC19 were plated onto brain heart infusion (BHI) agar containing appropriate antibiotics for selection. Cultures for invasion validation in this study were prepared from each of two isolated colony of each strain and grown to late log phase (OD₆₀₀0.8-1.0) with incubation at 37° C. in BHI medium with appropriate antibiotics.

A standard invasion assay was also used to demonstrate bacterial invasion of human alveolar basal epithelial cells (A549 cells), which are positive for both the HER2 receptor and furin.

Cells were then isolated and transferred to glass slides for imaging via laser scanning confocal microscopy. The cells were fixed in 10% NBF and mounted under a coverslip with Fluoromount-G mounting medium containing DAPI. The slides were imaged with a Zeiss LSM510 meta microscope, and images were collected at 40X magnification with an excitation wavelength of 488 nm.

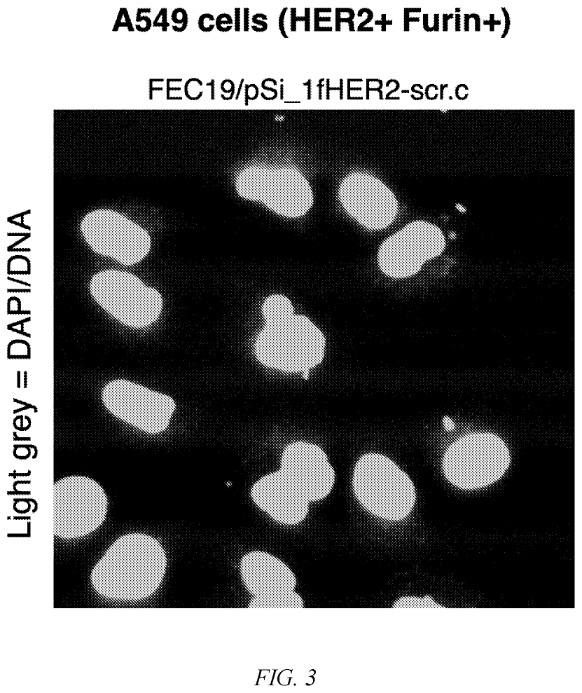

The micrograph in FIG. 3 shows successful invasion of FEC19/pSi_1fHER2-scr.c into A549 cells, thus demonstrating the function of this chimeric invasion targeting system. The FEC19 bacteria are visualized as the small bacillus-shaped grey masses near the larger grey masses, which are the nuclei of the eukaryotic cells.

In addition to natural binding domains, proteins and polypeptides with synthetic (i.e., non-natural) binding domains (e.g., single-domain antibodies or nanobodies) can be discovered using various computational and biochemical approaches. The use of synthetic binding domains designed to target a specific surface-presented binding ligand on the target cell surface will afford additional opportunities to target specific cell types. For example, as shown in the Example, a synthetic nanobody that binds to the human protein HER2, which is found on the surface of many cancer cells, was fused in frame to the full-length Inv sequence (D1-D5) to form a chimeric Inv protein that only invade HER2-positive (i.e., cells with HER2 on their surface) cells, in this example, A549 cells were used.

### Glossary of Claim Terms

The term “about” or “approximately” as used herein means within 20%, preferably within 10%, and more preferably within 5% of a given value or range.

The term “administration” and variants thereof (e.g., “administering” a compound) in reference to a compound of the invention means introducing the compound into the system of the subject in need of treatment. When a compound of the invention is provided in combination with one or more other active agents (e.g., an AIV vaccine, etc.), “administration” and its variants are each understood to include concurrent and sequential introduction of the compound and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to a viral infection, an effective amount comprises an amount sufficient to prevent contracting the disease or to reduce the severity of the disease as evidenced by clinical disease, clinical symptoms, viral titer or virus shedding from the subject, or as evidenced by the ability to prevent or reduce transmission between animals. In some embodiments, an effective amount is an amount sufficient to delay onset of clinical illness and/or symptoms or to prevent the disease. In some embodiments, an effective amount is an amount sufficient to lower viral titers and/or reduce viral shedding. An effective amount can be administered in one or more doses.

As used herein, “treatment” refers to obtaining beneficial or desired clinical results. Beneficial or desired clinical results include, but are not limited to, any one or more of: alleviation of one or more symptoms, diminishment of extent of viral infection, stabilized (i.e., not worsening) state of viral infection, preventing or delaying spread (e.g., shedding) of the viral infection, preventing, delaying or slowing of viral infection progression, and/or maintain weight/weight gain. The methods of the invention contemplate any one or more of these aspects of treatment.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, the term “invasive” when referring to a microorganism, e.g., a bacterium or bacterial therapeutic particle (BTP), refers to a microorganism that is capable of delivering at least one molecule, e.g., an RNA or RNA-encoding DNA molecule, to a target cell. An invasive microorganism can be a microorganism that is capable of traversing a cell membrane, thereby entering the cytoplasm of said cell, and delivering at least some of its content, e.g., RNA or RNA-encoding DNA, into the target cell. The process of delivery of the at least one molecule into the target cell preferably does not significantly modify the invasion apparatus.

As used herein, the term “transkingdom” refers to a delivery system that uses bacteria (or another invasive microorganism) to generate nucleic acids, proteins, antibodies, antibody derivatives, polypeptides, gene-editing systems (CRISPR and other gene editing nucleases), eukaryote-translatable mRNA or combinations thereof, and deliver the nucleic acids, proteins, antibodies, antibody derivatives, polypeptides, gene-editing systems (CRISPR and other gene editing nucleases), eukaryote-translatable mRNA or combinations thereofor intracellularly (i.e. across kingdoms: prokaryotic to eukaryotic, or across phyla: invertebrate to vertebrate) within target tissues for processing without host genomic integration.

Invasive microorganisms include microorganisms that are naturally capable of delivering at least one molecule to a target cell, such as by traversing the cell membrane, e.g., a eukaryotic cell membrane, and entering the cytoplasm, as well as microorganisms which are not naturally invasive and which have been modified, e.g., genetically modified, to be invasive. In another preferred embodiment, a microorganism that is not naturally invasive can be modified to become invasive by linking the bacterium or BTP to an “invasion factor”, also termed “entry factor” or “cytoplasm-targeting factor”. As used herein, an “invasion factor” is a factor, e.g., a protein or a group of proteins which, when expressed by a non-invasive bacterium or BTP, render the bacterium or BTP invasive. As used herein, an “invasion factor” is encoded by a “cytoplasm-targeting gene”. Invasive microorganisms have been generally described in the art, for example, U.S. Pat. Pub. Nos. US 20100189691 A1 and US 20100092438 A1 and Xiang, S. et al., Nature Biotechnology 24, 697-702 (2006). Each of which is incorporated by reference in its entirety for all purposes.

In a preferred embodiment the invasive microorganism is E. coli, as taught in the examples of the present application. However, it is contemplated that additional microorganisms could potentially be adapted to perform as transkingdom delivery vehicles for the delivery of NA. These non-virulent and invasive bacteria and BTPs would exhibit invasive properties, or would be modified to exhibit invasive properties, and may enter a host cell through various mechanisms. In contrast to uptake of bacteria or BTPs by professional phagocytes, which normally results in the destruction of the bacterium or BTP within a specialized lysosome, invasive bacteria or BTP strains have the ability to invade non-phagocytic host cells. Naturally occurring examples of such intracellular bacteria are Yersinia, Rickettsia, Legionella, Brucella, Mycobacterium, Helicobacter, Coxiella, Chlamydia, Neisseria, Burkolderia, Bordetella, Borrelia, Listeria, Shigella, Salmonella, Staphylococcus, Streptococcus, Porphyromonas, Treponema, and Vibrio, but this property can also be transferred to other bacteria or BTPs such as E. coli, Lactobacillus, Lactococcus, or Bifidobacteriae, including probiotics through the transfer of invasion-related genes (P. Courvalin, S. Goussard, C. Grillot-Courvalin, C. R. Acad. Sci. Paris 318, 1207 (1995)). Factors to be considered or addressed when evaluating additional bacterial species as candidates for use as transkingdom NA delivery vehicles include the pathogenicity, or lack thereof, of the candidate, the tropism of the candidate bacteria for the target cell, or, alternatively, the degree to which the bacteria can be engineered to deliver NA to the interior of a target cell, and any synergistic value that the candidate bacteria might provide by triggering the host's innate immunity.

Nucleic acids are defined as deoxyribonucleic acids (DNA), ribonucleic acids (RNA), or any closely related compound. They can be coding or non-coding, synthetically or naturally derived, single or double-stranded segments, often consist of molecules of many (2 or more) nucleotides linked. Examples include and are not limited to small interfering RNA/short hairpin RNA (siRNA/shRNA), micro RNA (miRNA), antagomiRs, RNA or DNA aptamers, messenger RNA (mRNA), splice-switching oligonucleotides, antisense oligonucleotides, antigene oligonucleotides, DNAzymes, RNA decoys, ribozymes, peptide nucleic acids, oligomers, and defective interfering particles.

Therapeutic nucleic acids are NAs as described herein or a closely related chemical compound used to treat disease, study disease, or used to achieve a desired genetic modification or used for gene transfer purposes. They are used in cases where specific inhibition or interruption or altering of the function of a particular gene or other molecule involved in disease is thought to be therapeutically desirable.

Synthetic binding proteins are human-made proteins that have been tailored to bind to a target molecule of interest. Synthetic binding domains are the binding domain of a synthetic binding protein. Synthetic binding proteins (SBPs) are smaller, more stable, less immunogenic, and better of tissue penetration than typical non-synthetic alternatives. SBPs include affibodies, anticalins, DARPins, i-bodies, monobodies/adnectins, nanobodies, repebodies, scFabs, scFvs and vNARs. It is contemplated that SBPs and/or their binding domain, including the aforementioned, can be utilized in a chimeric invasin polypeptide. SBPs are discussed in Sha F. Salzman G, Gupta A, Koide S. Monobodies and other synthetic binding proteins for expanding protein science. Protein Sci. 2017 May;26(5):910-924. doi: 10.1002/pro.3148. Epub 2017 Mar. 24. PMID: 28249355; PMCID: PMC5405424 and Xiaona Wang, Fengcheng Li, Wengi Qiu, Binbin Xu, Yanlin Li, Xichen Lian, Hongyan Yu, Zhao Zhang, Jianxin Wang, Zhaorong Li, Weiwei Xue, Feng Zhu, SYNBIP: synthetic binding proteins for research, diagnosis and therapy, Nucleic Acids Research, Volume 50, Issue D1, 7 Jan. 2022, Pages D560-D570, https://doi.org/10.1093/nar/gkab926.

A nanobody, also known as a single-domain antibody (sdAb), is an antibody fragment consisting of a single monomeric variable antibody domain.

Affibody molecules are small, robust proteins engineered to bind to a large number of target proteins or peptides with high affinity, imitating monoclonal antibodies, and are therefore a member of the family of antibody mimetics. These molecules can be used for molecular recognition in diagnostic and therapeutic applications.

DARPins (designed ankyrin repeat proteins) are genetically engineered antibody mimetic proteins typically exhibiting highly specific and high-affinity target protein binding. They are derived from natural ankyrin repeat proteins, one of the most common classes of binding proteins in nature, which are responsible for diverse functions such as cell signaling, regulation and structural integrity of the cell. DARPins consist of at least three, repeat motifs or modules, of which the most N-and the most C-terminal modules are referred to as “caps”, since they shield the hydrophobic core of the protein.

Anticalin proteins are artificial proteins that are able to bind to antigens, either to proteins or to small molecules. They are not structurally related to antibodies, which makes them a type of antibody mimetic. Instead, they are derived from human lipocalins which are a family of naturally binding proteins. Anticalin proteins are being used in lieu of monoclonal antibodies, but are about eight times smaller with a size of about 180 amino acids and a mass of about 20 kDa.

As used herein, a disease is prevented before or after exposure to the disease, if (1) a medicament composition is administered to a subject internally (by ingestion, inhalation, injection, etc.), topically (on the skin for absorption into the body), or otherwise, and (2) the medicament composition prevents the subject from contracting the disease and experiencing symptoms/clinical illness normally associated with the disease, or, if the subject contracts the disease and experiences or doesn't experience in varying degrees of severity some or all of the symptoms/clinical disease normally associated with the disease, the subject recovers from the disease to a normal healthy state.

Kits for practicing the methods of the invention are further provided. By “kit” is intended any manufacture (e.g., a package or a container) comprising at least one reagent, e.g., a pH buffer of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

In an advantageous embodiment, the kit containers may further include a pharmaceutically acceptable carrier. The kit may further include a sterile diluent, which is preferably stored in a separate additional container. In another embodiment, the kit further comprising a package insert comprising printed instructions directing the use of a combined treatment of a pH buffer and the anti-pathogen agent as a method for treating and/or preventing disease in a subject. The kit may also comprise additional containers comprising additional anti- pathogen agents (e.g. amantadine, rimantadine and oseltamivir), agents that enhance the effect of such agents, or other compounds that improve the efficacy or tolerability of the treatment. A kit could also include at least one reagent that is used to perform a particular conventional technique that are within the skill of the art (i.e. nucleic acid extraction).

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman,Proc. Natl. Acad. Sci. USA 85:2444, 1988; Higgins & Sharp, Gene, 73:237-44, 1988; Higgins & Sharp, Comput. Appl. Biosci. 5:151-3, 1989; Corpet et al., Nucl. Acids Res. 16:10881-90, 1988; Huang et al. Comput. Appl. Biosci. 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the examples herein above. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, POR Primer: A Laboratory Manual, and Molecular Cloning: A Laboratory Manual (all from Cold Spring Harbor Laboratory Press), Stryer, L. (1995) Biochemistry (4th Ed.) Freeman, N. Y., Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

### REFERENCES

Isberg et al. 1987: Isberg R R, Voorhis D L, Falkow S. Identification of invasin: a protein that allows enteric bacteria to penetrate cultured mammalian cells. Cell. 1987 Aug. 28;50(5):769-78. doi: 10.1016/0092-8674(87)90335-7. PMID: 3304658.
Chain et al. 2004: Chain P S, Carniel E, Larimer F W, Lamerdin J, Stoutland P O, Regala W M, Georgescu A M, Vergez L M, Land M L, Motin V L, Brubaker R R, Fowler J, Hinnebusch J, Marceau M, Medigue C, Simonet M, Chenal-Francisque V, Souza B, Dacheux D, Elliott J M, Derbise A, Hauser L J, Garcia E. Insights into the evolution of Yersinia pestis through whole-genome comparison with Yersinia pseudotuberculosis. Proc Natl Acad Sci U S A. 2004 Sep 21;101(38):13826-31. doi: 10.1073/pnas.0404012101. Epub 2004 Sep. 9. PMID: 15358858; PMCID: PMC518763.
Leong et al. 1990: Leong J M, Fournier R S, Isberg R R. Identification of the integrin binding domain of the Yersinia pseudotuberculosis invasin protein. EMBO J. 1990 June;9 (6): 1979-89. PMID: 1693333; PMCID: PMC551907.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

TABLE 1
Bacteria-derived heterologous proteins with binding domains.

| | Bacterial | |
|---|---|---|
| Protein | Species | Receptor(s) |
| FimH | Escherichia | mannosides, laminin, fibronectin, plasminogen |
| | coli | |
| | Klebsiella | mannosides, laminin, fibronectin, plasminogen |
| | pneumoniae | |
| | Salmonella | mannosides, laminin, fibronectin, plasminogen |
| | spp. | |
| PapG | E. coli | Gala(1-4)Gal moiety in globoseries of glycolipids |
| PrsG | E. coli | Gala(1-4)Gal moiety in globoseries of glycolipids |
| SfaS | E. coli | a-sialyl-(2-3)- b-galactose |
| FocH | E. coli | N-acetylgalactosamine, galactose, glycophorin |
| FimD | B. pertussis | unknown |
| MrpH | Proteus | Gala(1-4)Gal moiety in globoseries of glycolipids |
| | mirabilis | |
| FaeG | E. coli | Gala (1-3)Gal, Galb, GlcNAc, GaINAc, fucose, |
| | | polymycin B, nonapeptide |
| FanC | E. coli | NeuGe-GM3, NeuGc-SPG, sialoglycoproteins |
| CfaB | E. coli | NeuAc-GM2, human erythrocyte |
| | | sialylglycoprotein, HT-29 glycoprotein |
| MrkD | K. | type V collagen |
| | pneumoniae | |
| PilC | Neisseria | unknown |
| | spp. | |
| CsgA | E. coli | fibronectin, plasminogen, human contact phase |
| | | proteins |
| Afa-IE | E. coli | determinants of the Dr(a) blood group marker |
| | | DAF |
| DraA | E. coli | determinants of the Dr(a) blood group marker |
| NfaA | E. coli | carbohydrate moieties associated with |
| | | glycophorin A |
| AIDA-1 | E. coli | HEp-2 and HeLa cells |
| Intimin | E. coli | Tir protein |
| alpha | | |

TABLE 2
Fungus-derived heterologous proteins with binding domains.

| Protein | Fungal species | Receptor(s)/Target |
|---|---|---|
| RodA | Aspergillus fumigatus | Collagen |
| Mp1 (galactomannoprotein) | A. fumigatus | ECM |
| Extracellular Thaumatin | A. fumigatus | Laminin, Fibrinogen, Mice Lung |
| domain protein (AfCalAp) | | cells |
| Hydrophobins | A. fumigatus | ECM |
| CspA | A. fumigatus | ECM |
| Alpha-mannosidase | A. fumigatus | Fibrinogen |
| Glyceraldehyde-3- | Candida albicans | fibronectin and laminin |
| phosphatedehydrogenase | | |
| (GAPDH) | | |
| Als1p | C. albicans | Yeast form |
| Hwp1 | C. albicans | Mammalian transglutaminases |
| EAP1g | C. albicans | Epithelial cells |
| Int1 | C. albicans | ECM proteins |
| ALS (Als 1p-Als9p) | C. albicans | ECM proteins |
| CaIff4 | C. albicans | Epithelial cells; plastic surface |
| Alcohol dehydrogenase | C. albicans | Vitronectin, fibronectin, laminin |
| (Adh1) | | |
| Phosphoglycerate mutase | C. albicans | Vitronectin |
| (Gpm1) | | |
| Epa1p/EPA1g | Candida glabrata | Host-cell carbohydrates |
| Surface expressed integrin | Candida tropicalis | Fibronectin |
| analogue (putative fibronectin | | |
| receptor) | | |
| Surface Als like proteins | Candida parapsilosis | ECM proteins |
| SOWgpg/rSOWp | Coccidioides immitis | ECM proteins |
| Heat shock protein (Hsp60) | C. immitis | CD18 receptors on macrophage |
| | | cells, CHO cells |
| MAD1 and MAD2 | Metarhizium anisopliae | ECM |
| Cell Wall Mannoprotein | Penicillium marneffei | Concanavalin A (a type of lectin |
| Mp1p (58-kDa) | | purified from jack beans, binds with |
| | | mannse residues of glycoproteins) |
| Glyceraldehyde-3- | P. marneffei | A549 pneumocytes, fibronectin and |
| phosphatedehydrogenase | | laminin |
| (GAPDH) | | |
| Glyceraldehyde-3-phosphate | Paracoccidioides | fibronectin, laminin, and type I |
| dehydrogenase (GAPDH) | brasiliensis | collagen |
| Pb 14-3-3 protein | P. brasiliensis | S. cerevisiae cells expressing Pb 14- |
| | | 3-3 adhere to epithelial cell-line |
| | | A549 |
| Peptidorhamnomannan | Pseudallescheria boydii | HEp2 cells (human larynx |
| (PRM) | | carcinoma cells) |
| Msg protein | Pneumocystis jirovecii | A549 cell-line |

TABLE 3
Virus-derived heterologous proteins with binding domains.

| Protein | Virus | Receptor(s) |
|---|---|---|
| | Myxoviruses | |
| hemagglutinin | Influenza A and B | Neu5Acα2-6Gal- |
| | (human, ferret, and | |
| | porcine) | |
| hemagglutinin | Influenza A and B | Neu5Acα2-3Gal- |
| | (avian and porcine) | |
| hemagglutinin- | Influenza C | 9-O-acetyl-Siaα- |
| esterase | | |
| hemagglutinin- | Newcastle disease | Neu5Acα2-3Gal- |
| neuraminidase | | |
| hemagglutinin- | Sendai | Neu5Acα2-8Neu5Ac- |
| neuraminidase | | |
| | Polyomaviruses | |
| capsid protein | Polyoma | Neu5Acα2-3Gal-, Neu5Acα2- |
| VP1 | | 3Galβ1-3 (Neu5Acα2-6)GalNAc |
| | | on gangliosides such as GM1 and |
| | | GT1b/GD1a |
| | Herpesviruses | |
| glycoproteins | Herpes simplex | 3-O-sulfated heparan sulfate |
| gB, gC, and gD | | |
| | Picornaviruses | |
| caspid proteins | Foot-and-mouth | heparan sulfate |
| | disease (enterovirus) | |
| | Retroviruses | |
| gp120 V3 loop | HIV | heparan sulfate |
| | Flaviviruses | |
| envelope | Dengue | heparan sulfate |
| protein | | |
| | Calciviruses | |
| capsid proteins | Norovirus | fucose, GalNAc, or Gal on A and |
| | | B blood group antigens |

TABLE 4
Peptidases and proteases.

| Name | Alternative Name | Substrate |
|---|---|---|
| Aminopeptidase N | APN (CD13) | N-terminal amino acid residues |
| Aminopeptidase A | APA | N-terminal acidic amino acids |
| Aminopeptidase P | APP | N-terminal amino acid linked with |
| | | proline |
| Dipeptidyl peptidase 9 | DPP9 | N-terminal X-proline dipeptides |
| Pyroglutamyl-peptidase II | TRHDE | N-terminal pyroglutamyl group from |
| | | pGlu--His-Xaa tripeptides and pGlu-- |
| | | His-Xaa-Gly tetrapeptides |
| Dipeptidyl-peptidase IV | DPP IV (CD26) | N-terminal X-proline or X-alanine |
| | | dipeptides |
| Angiotensin-converting enzyme | ACE (CD143) | C-terminal dipeptides |
| Angiotensin-converting enzyme-2 | ACE2, ACEH | Asp-Arg-Val-Tyr-Ile-His-Pro-Phe [SEQ. |
| | | ID. NO. 21] |
| Carboxypeptidase M | CPM | C-terminal arginine or lysine from |
| | | polypeptides |
| Carboxypeptidase P | CPP | C-terminal amino acids |
| γ-Glutamyl transpeptidase | γ-GT (CD224) | |
| Membrane dipeptidase | MDB | |
| Neprilysin | NEP (CD10) | |
| Endothelin-converting enyzme | ECE-1 | XXXFLVXXX [SEQ. ID. NO. 22] |
| Prostasin | PRSS8 | |
| Matriptase | ST14 | RXXRKVXG [SEQ. ID. NO. 23]; |
| | | AVIGRKFGDP [SEQ. ID. NO. 24] |
| Matriptase-2 | TMPRSS6 | XXXRKXXX [SEQ. ID. NO. 25]; |
| | | AVIGRKFGDP [SEQ. ID. NO. 26] |
| Matriptase-3 | TMPRSS7 | XXXRKXXX [SEQ. ID. NO. 27]; |
| | | AVIGRKFGDP [SEQ. ID. NO. 28] |
| Polyserase-1 | TMPRSS9 | — |
| Transmembrane protease, serine 2 | TMPRSS2 | — |
| Transmembrane protease, serine 3 | TMPRSS3 | — |
| Transmembrane protease, serine 4 | TMPRSS4 | RXXRLXXEE [SEQ. ID. NO. 29] |
| Transmembrane protease, serine | TMPRSS5 | — |
| 5/Spinesin | | |
| Corin | CORIN | |
| Furin | FURIN | RXXR |
| DPPIV | DPP4 | N-terminal X-P X-A dipeptides |
| MT1-MMP | MMP14 | PENFFG [SEQ. ID. NO. 30]; |
| | | TSEDFLVVQ [SEQ. ID. NO. 31] |
| MT4-MMP | MMP17 | |
| Gelatinase | MMP2; MMP9 | PENFFG [SEQ. ID. NO. 32]; |
| | | TSEDFLVVQ [SEQ. ID. NO. 33] |
| A Disintegrin and metalloproteinase | ADAM8 | |
| domain-containing protein 8 | | |
| A Disintegrin and metalloproteinase | ADAM9 | |
| domain-containing protein 9 | | |
| A Disintegrin and metalloproteinase | ADAM10 | |
| domain-containing protein 10 | | |
| A Disintegrin and metalloproteinase | ADAM12 | |
| domain-containing protein 12 | | |
| A Disintegrin and metalloproteinase | ADAM15 | |
| domain-containing protein 15 | | |
| A Disintegrin and metalloproteinase | ADAM17 | |
| domain-containing protein 17 | | |
| A Disintegrin and metalloproteinase | ADAM33 | |
| domain-containing protein 33 | | |
| Hepsin | HPN | AVIGRKFGDP [SEQ. ID. NO. 34] |
| Fibroblast activation protein | FAP | |
| Neutral endopeptidase/Neprilysin | MMEL1 | |
| Meprin A | MEP1A | |
| Meprin B | MEP1B | |
| Testisin | PENFFG; | |
| | TSEDFLVV | |
| HAT | HAT | AVIGRKFGDP [SEQ. ID. NO. 35] |
| Transmembrane protease serine 11E | DESC1 | AVIGRKFGDP [SEQ. ID. NO. 36] |
| Transmembrane protease serine 11A | TMPRSS11A | |
| Transmembrane protease serine 11F | TMPRSS11F | |
| Transmembrane protease serine 11B | TNPRSS11B | |
| MSPL | TMPRSS13 | |
| Enteropeptidase | TMPRSS15 | |
| Insulin regulated aminopeptidase | IRAP | |

1. An expression cassette for the production of a chimeric invasin (Inv) polypeptide comprising a prokaryotic promoter and a nucleic acid sequence encoding an Inv polypeptide fused to a linker polypeptide at the carboxy terminus of the Inv polypeptide and wherein expression of the nucleic acid encoding the chimeric invasin polypeptide is controlled by the prokaryotic promoter.

2. The expression cassette for the production of a chimeric invasin polypeptide according to claim 1 further comprising a sequence encoding a binding domain wherein the binding domain is fused to the amine terminus of the linker polypeptide.

3. The expression cassette for the production of a chimeric invasin polypeptide according to claim 2 wherein the binding domain is a binding domain from a protein listed in Tables 1-3 or the binding domain is a synthetic binding domain.

4. The expression cassette for the production of a chimeric invasin polypeptide according to claim 1 wherein the nucleic acid sequence of the invasin region of the chimeric polypeptide is 90% identical to nucleic acids 1-2958 of SEQ ID. NO. 37.

5. The expression cassette for the production of a chimeric invasin polypeptide according to claim 1 wherein the nucleic acid sequence of the invasin region of the chimeric polypeptide is 95% identical to nucleic acids 1-2958 of SEQ ID. NO. 37.

6. The expression cassette for the production of a chimeric invasin polypeptide according to claim 1 wherein the nucleic acid sequence of the invasin region of the chimeric polypeptide is 99% identical to nucleic acids 1-2958 of SEQ ID. NO. 37.

7. The expression cassette for the production of a chimeric invasin polypeptide according to claim 1 wherein the nucleic acid sequence of the invasin region of the chimeric polypeptide is 90% identical to nucleic acids 1-2382 of SEQ ID. NO. 37 or SEQ ID. NO. 38.

8. The expression cassette for the production of a chimeric invasin polypeptide according to claim 1 wherein the nucleic acid sequence of the invasin region of the chimeric polypeptide is 95% identical to nucleic acids 1-2382 of SEQ ID. NO. 37.

9. The expression cassette for the production of a chimeric invasin polypeptide according to claim 1 wherein the nucleic acid sequence of the invasin region of the chimeric polypeptide is 99% identical to nucleic acids 1-2382 of SEQ ID. NO. 37.

10. The expression cassette for the production of a chimeric invasin polypeptide according to claim 1 wherein the nucleic acid sequence encodes a linker polypeptide selected from the group consisting of [SEQ. ID. NO. 2] through [SEQ ID. NO. 20] and/or a protease cleavage site selected from the group consisting of [SEQ. ID. NO. 22] through [SEQ ID. NO. 36].

11. The expression cassette for the production of a chimeric invasin polypeptide according to claim 1 wherein the nucleic acid sequence encodes a protease cleavage site cleaved by a peptidase or protease.

12. The expression cassette for the production of a chimeric invasin polypeptide according to claim 1 wherein the nucleic acid sequence encodes a protease cleavage site cleaved by a peptidase or protease listed in Table 4.

13. The expression cassette for the production of a chimeric invasin polypeptide according to claim 2 wherein the nucleic acid sequence encoding the protease cleavage site cleaved by a peptidase or protease is between the sequence encoding the invasin and the sequence encoding the binding domain.

14. The expression cassette for the production of a chimeric invasin polypeptide according to claim 1 wherein the prokaryotic promoter is a promoter selected from the group consisting of T7, lacUV5, gapA, T5, recA, Ptac, Patac, pAl, lac, Sp6, araBad, and trp.

15. The expression cassette for the production of a chimeric invasin polypeptide according to claim 1 wherein the prokaryotic promoter is a hybrid or synthetic prokaryotic promoter.

16. (canceled)

17. The bacterium expressing a chimeric invasin polypeptide according to claim 16 claim 1 wherein the bacterium is a bacterium selected from the group consisting of Clostridium difficile, Escherichia coli, Clostridium tetani, Helicobacter pylori, Fusobacterium nucleatum, Gardnerella vaginitis, Porphyromonas gingivalis, Aggregatibacter actinomycetemcomitans, Listeria monocytogenes, Staphylococcus aureus, Campylobacter jejuni, Vibrio vulnificus, Salmonella typhi, Clostridium botulinum, Mycobacterium tuberculosis, Mycobacterium leprae, Mycobacterium lepromatosis, Corynebacterium diptheriae, Klebsiella pneumoniae, Acinetobacter baumannii, Streptococcus mutans, group B streptococci, Staphylococcus aureus, Streptococcus agalactiae, Streptococcus pneumonia, Enterococcus spp., Enterococcus faecalis, Listeria, Yersinia, Rickettsia, Shigella, Salmonella spp., Legionella, Chlamydia, Brucella, Neisseria, Burkolderia, Bordetella, Borrelia, Coxiella, Mycobacterium, Helicobacter, Staphylococcus, Streptococcus, Porphyromonas, Vibrio, Treponema, Lactobacillus, and Bifidobacteriae.

18. The bacterium expressing a chimeric invasin polypeptide according to claim 1 wherein the bacterium is an Escherichia coli bacterium.

19. (canceled)

20. (canceled)

21. A chimeric invasin polypeptide comprising an Inv polypeptide and a linker polypeptide, wherein the linker polypeptide has a first end (N-terminus) and a second end (C-terminus), wherein the first end (N-terminus) of the linker polypeptide is attached to the C-terminus of the Inv polypeptide.

22-31. (canceled)

32. A chimeric invasin polypeptide comprising the D1-D3 domains of the Inv polypeptide attached to the binding domain of a heterologous protein or a synthetic binding domain.

33-65. (canceled)


SEQUENCE LISTING (42 sequences)

SEQ ID NO: 1 - protein, 986 aa, Yersinia pseudotuberculosis
MMVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAVPTSIIADGLMASTITLELKDTYGDPQAGANVAFDTTLGNMGVITDHNDGTYSAPLTSTTLGVATVTVKVDGAAFSVPSVTVNFTADPIPDAGRSSFTVSTPDILADGTMSSTLSFVPVDKNGHFISGMQGLSFTQNGVPVSISPITEQPDSYTATVVGNSVGDVTITPQVDTLILSTLQKKISLFPVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGRSLVSSLEASRQCQGSDMSAVLESSRATNGTRAPDGTLWGEWGSLTAYSSDWQSGEYWVKKTSTDFETMNMDTGALQPGPAYLAFPLCALSI

SEQ ID NO: 2 - protein, 10 aa, synthetic construct
EAAAREAAAR

SEQ ID NO: 3 - protein, 20 aa, synthetic construct
EAAAREAAAREAAAREAAAR

SEQ ID NO: 4 - protein, 6 aa, synthetic construct
GSGSGS

SEQ ID NO: 5 - protein, 10 aa, synthetic construct
GSGSGSGSGS

SEQ ID NO: 6 - protein, 4 aa, synthetic construct
GGGS

SEQ ID NO: 7 - protein, 5 aa, synthetic construct
GGGGS

SEQ ID NO: 8 - protein, 13 aa, synthetic construct
GGGSGGGGSGGGS

SEQ ID NO: 9 - protein, 4 aa, synthetic construct
GGSG

SEQ ID NO: 10 - protein, 8 aa, synthetic construct
GGSGGGSG

SEQ ID NO: 11 - protein, 12 aa, synthetic construct
GGSGGGSGGGSG

SEQ ID NO: 12 - protein, 5 aa, synthetic construct
GSGGS

SEQ ID NO: 13 - protein, 5 aa, synthetic construct
GSSGS

SEQ ID NO: 14 - protein, 10 aa, synthetic construct
ACGSLSCGSF

SEQ ID NO: 15 - protein, 10 aa, synthetic construct
GENLYFQSGG

SEQ ID NO: 16 - protein, 8 aa, synthetic construct
SACYCELS

SEQ ID NO: 17 - protein, 17 aa, synthetic construct
RPACKIPNDLKQKVMNH

SEQ ID NO: 18 - protein, 12 aa, synthetic construct
PPPYQPLGGGGS

SEQ ID NO: 19 - protein, 51 aa, synthetic construct
WRKRLRKKRLRKKRRLKKRRRKKQRRKRRLEGSGQGPGSGQGSGSPGSGQG

SEQ ID NO: 20 - skipped

SEQ ID NO: 21 - protein, 8 aa, synthetic construct
DRVYIHPF

SEQ ID NO: 22 - skipped

SEQ ID NO: 23 - protein, 8 aa, synthetic construct
RXXRKVXG

SEQ ID NO: 24 - protein, 10 aa, synthetic construct
AVIGRKFGDP

SEQ ID NO: 25 - skipped

SEQ ID NO: 26 - protein, 10 aa, synthetic construct
AVIGRKFGDP

SEQ ID NO: 27 - skipped

SEQ ID NO: 28 - protein, 10 aa, synthetic construct
AVIGRKFGDP

SEQ ID NO: 29 - protein, 9 aa, synthetic construct
RXXRLXXEE

SEQ ID NO: 30 - protein, 6 aa, synthetic construct
PENFFG

SEQ ID NO: 31 - protein, 9 aa, synthetic construct
TSEDFLVVQ

SEQ ID NO: 32 - protein, 6 aa, synthetic construct
PENFFG

SEQ ID NO: 33 - protein, 9 aa, synthetic construct
TSEDFLVVQ

SEQ ID NO: 34 - protein, 10 aa, synthetic construct
AVIGRKFGDP

SEQ ID NO: 35 - protein, 10 aa, synthetic construct
AVIGRKFGDP

SEQ ID NO: 36 - protein, 10 aa, synthetic construct
AVIGRKFGDP

SEQ ID NO: 37 - DNA, 2958 bp, Yersinia pseudotuberculosis
  source 1..2958: mol_type = other DNA
atgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaata

SEQ ID NO: 38 - DNA, 2382 bp, Yersinia pseudotuberculosis
  source 1..2382: mol_type = other DNA
atgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccg

SEQ ID NO: 39 - protein, 985 aa, Yersinia pseudotuberculosis
MVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAVPTSIIADGLMASTITLELKDTYGDPQAGANVAFDTTLGNMGVITDHNDGTYSAPLTSTTLGVATVTVKVDGAAFSVPSVTVNFTADPIPDAGRSSFTVSTPDILADGTMSSTLSFVPVDKNGHFISGMQGLSFTQNGVPVSISPITEQPDSYTATVVGNTAGDVTITPQVDTLILSTLQKKISLFPVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGTSLVSSLEASRQCQGSDMSAVLESSRATNGTRAPDGTLWGEWGSLTAYSSDWQSGEYWVKKTSTDFETMNMDTGALVQGPAYLAFPLCALAI

SEQ ID NO: 40 - DNA, 9275 bp, synthetic construct
  source 1..9275: mol_type = other DNA
taatagaaaaaaaacgttaccacagtgctttcgcactgtggtaacgttttttttccagataacagatagcaataagaacagtttaatgagctgattatttggggcgcgaatgggagtccggcaatcctagactcgccccataagtagcaaacgtccagaagaacaacgccgctcaggttaattgagcggcgctgtttttttaaaaggattgtcgcgataagcgtgagctggcgttaaatgccgatcttacggcccagctgcagcccgggggatctatgcggtgtgaaataccgcacagatgcgtaaggagaaaataccgcatcaggcgccattcgccattcaggctgcgcaactgttgggaagggcgatcggtgcgggcctcttcgctattacgccaggacttcatatacccaagcttggaaaattttttttaaaaaagtcttgacactttatgcttccggctcgtataatggatccatatgcggccgcatatggatccatatgcggcagcatgcagatctagactagtcgacgatccttagcgaaagctaaggattttttttttactcgagcggattactacatacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgatctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgacggtatcgataagcttgataagcttttaaatcagcaggggtctttttggcttgtgtattattttgaagtttttcttccccgacagaatctgcttttaccgtcatagtgaaatgagcctgaaaagctattaccatgatgatacaaataagtttacttttcatttcaccgctcctttttaattcgtaaaactaagtttaagccacctacaactaatctgacagagagagttaaggacacgttttttagtatatgtgggaactaaattatacgttttgcagtagaaactataggtggcttaaactttgggatatgcttatattatatggataaacagtcagatattcttttacatttgttaattcttctaaaaaaattaaaaaataagcctgtttctacattcttcacaaaataatttacgaagagtgcaaaacaagcttattttttcgtgtgtgttaagcggttttattcttaattttttattacttttacaattattcgattggattatctactttattactatatttcggataaagcgtggtgccccagatggagatatttctatttttcacaagtggtaagttccggtcatcaattaccgttctccaccattcccaagctaaaccagtgcattctttagcgtaaacattaatatttctcgcgttacctggcaaatagatggacgatgtgaaatgagctagcttgcttttattgttttcgctccagtttttatgttgaacaatttcgttaccttcaggatcataatttacttcatcccaagaaatgttgaattgagcaacgtatcctccagagtgatcgatgttaatttttccatctgtataagcttttgaagttgtttcaatatattctgagttgtttttaataacagctaattcattgtcttttaggaagtttgttgtataagcaatgggaactcctggtgtttctcgattaaaagtagcgccttttttcaaaatatcgcgtaagtctccgaggttgccgtcgatgatttgaacttcatcttttgcggaacctccgtaaattacggctttgaaggaagaatttttgatgatatttgttagttctacatcacctgagacagattttccgcttacggcagcatcaaaagcagcttttactttagtactatgggaattagttgataatttcaaataaacttgacggccatacgccacacttgagatatatgcaggaggattttctgcattcactccaagcgcttgcaactgctctttagtaacagctttgccgaaaaatctggaaggtcttgtaggttcattaacattcacgttatagtaaatttgtttaaaactaatgacttcttcttgcattttcccttcactgattgcgccgaagtttacattcaagctattatttacagctttaaatgctgtaccaaatttcgcaattaattgtgattcactgtaagccatttcgtcatcataatcaatttttgcacttacatttggataagcttgagcatatttttcattccatctttccactaatgtatttactgcgttgttaacgtttgatttagtggcattttttacaacgattttattgtcttgattagtcatacctggcaaatcaatgctgagtgttaatgaatcacgttttacagggagaacatctggttgattttctactaattccgaattcgcttttacgagagcacctggataggttaggctcgaaattgcattcacaacttgaatgtctgcattattttgattgatggatttcttctttttctccacaacaatatattcatttccatctttgtaaccttttcttggcggcacatttgtcactgcatctccgtggtatactaatacattgtttttattgtaatccaatccttgtatatacttatcgatttcatccgcgtgtttcttttcgattggcgtcttaggacttgcaggcggagatgctggtggtgccatggatgaaattgaattttctttattgaatgcagatgcatcctttgcttcagtttgttgcgcaattggtagactaactaatataagtgtaataaaaactagcattatttttttcatgggtttcactctccttctacattttttaacctaataatgccaaataccgtttgccacccctctcttttgataattataatattggcgaaattcgcttctaaagatgaaacgcaatattatatgcttgctttatagctttattctagtcctgctgtccctttatcgtcgttaacaaatgttaatgcctcaacataaaagtcactttaagataggaatatactaatcaaaggagggatcgaattcctgcagtcatcaaggcaaccatcaggattaatgcggatattgcggagtaacacttcagactgaaagtagaaataaaaaccgcagcagacaactgacaacatcaaatgaagggggcttattctaattgatattatttatatgataatagttcattttgtatttttgttttttttgatattctcacctgcttagttacaataaatcaattctatcgctgtatggtatagactgttttattatatattttgaatatttttaatctgcccagtctggttttttaaaaaagtgctatcctcttaatgtctttactaaattagaaaacaagtttcactttcaactattgcatctttaattaatggtcaaggtgatttcaaatgctcgtttgtggccagttatacctcaaataactcaagttgttgagcacagccaacgcacatgcagtttgacgtatgacaggtatgctttatttcatttaaattatgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatacgccgcgcgcgcagcgtggcgagcatggaagttcagctggttgaatctggtggtggtctggttcaggcgggtggttctctgcgtctgtcttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttcaccatctctcgtgacaacgcgaaaaacaccgtttacctgcagatgaactctctgaaaccggaagacaccgcggtttactactgcaaacgtttccgtaccgcggcgcagggtaccgactactggggtcagggtacccaggttaccgtttcttctcaccaccaccaccaccacggtagcggcagcggtagcatggaagttcagctggttgaatctggtggtggtctggttcaggcgggtggttctctgcgtctgtcttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttcaccatctctcgtgacaacgcgaaaaacaccgtttacctgcagatgaactctctgaaaccggaagacaccgcggtttactactgcaaacgtttccgtaccgcggcgcagggtaccgactactggggtcagggtacccaggttaccgtttcttctcaccaccaccaccaccac

SEQ ID NO: 41 - DNA, 3363 bp, synthetic construct
  source 1..3363: mol_type = other DNA
atgatggttttccagccaatcagtgagtttctcttgataaggaatgcgggaatgtctatgtattttaataaaataatttcatttaatattatttcacgaatagttatttgtatctttttgatatgtggaatgttcatggctggggcttcagaaaaatatgatgctaacgcaccgcaacaggtccagccttattctgtctcttcatctgcatttgaaaatctccatcctaataatgaaatggagagttcaatcaatcccttttccgcatcggatacagaaagaaatgctgcaataatagatcgcgccaataaggagcaggagactgaagcggtgaataagatgataagcaccggggccaggttagctgcatcaggcagggcatctgatgttgctcactcaatggtgggcgatgcggttaatcaagaaatcaaacagtggttaaatcgattcggtacggctcaagttaatctgaattttgacaaaaatttttcgctaaaagaaagctctcttgattggctggctccttggtatgactctgcttcattcctcttttttagtcagttaggtattcgcaataaagacagccgcaacacacttaaccttggcgtcgggatacgtacattggagaacggttggctgtacggacttaatactttttatgataatgatttgaccggccacaaccaccgtatcggtcttggtgccgaggcctggaccgattatttacagttggctgccaatgggtattttcgcctcaatggatggcactcgtcgcgtgatttctccgactataaagagcgcccagccactgggggggatttgcgcgcgaatgcttatttacctgcactcccacaactgggggggaagttgatgtatgagcaatacaccggtgagcgtgttgctttatttggtaaagataatctgcaacgcaacccttatgccgtgactgccgggatcaattacacccccgtgcctctactcactgtcggggtagatcagcgtatggggaaaagcagtaagcatgaaacacagtggaacctccaaatgaactatcgcctgggcgagagttttcagtcgcaacttagcccttcagcggtggcaggaacacgtctactggcggagagccgctataaccttgtcgatcgtaacaataatatcgtgttggagtatcagaaacagcaggtggttaaactgacattatcgccagcaactatctccggcctgccgggtcaggtttatcaggtgaacgcacaagtacaaggggcatctgctgtaagggaaattgtctggagtgatgccgaactgattgccgctggcggcacattaacaccactgagtaccacacaattcaacttggttttaccgccttataaacgcacagcacaagtgagtcgggtaacggacgacctgacagccaacttttattcgcttagtgcgctcgcggttgatcaccaaggaaaccgatctaactcattcacattgagcgtcaccgttcagcagcctcagttgacattaacggcggccgtcattggtgatggcgcaccggctaatgggaaaactgcaatcaccgttgagttcaccgttgctgattttgaggggaaacccttagccgggcaggaggtggtgataaccaccaataatggtgcgctaccgaataaaatcacggaaaagacagatgcaaatggcgtcgcgcgcattgcattaaccaatacgacagatggcgtgacggtagtcacagcagaagtggaggggcaacggcaaagtgttgatacccactttgttaagggtactatcgcggcggataaatccactctggctgcggtaccgacatctatcatcgctgatggtctaatggcttcaaccatcacgttggagttgaaggatacctatggggacccgcaggctggcgcgaatgtggcttttgacacaaccttaggcaatatgggcgttatcacggatcacaatgacggcacttatagcgcaccattgaccagtaccacgttgggggtagcaacagtaacggtgaaagtggatggggctgcgttcagtgtgccgagtgtgacggttaatttcacggcagatcctattccagatgctggccgctccagtttcaccgtctccacaccggatatcttggctgatggcacgatgagttccacattatcctttgtccctgtcgataagaatggccattttatcagtgggatgcagggcttgagttttactcaaaacggtgtgccggtgagtattagccccattaccgagcagccagatagctataccgcgacggtggttgggaatagtgtcggtgatgtcacaatcacgccgcaggttgataccctgatactgagtacattgcagaaaaaaatatccctattcccggtacctacgctgaccggtattctggttaacgggcaaaatttcgctacggataaagggttcccgaaaacgatctttaaaaacgccacattccagttacagatggataacgatgttgctaataatactcagtatgagtggtcgtcgtcattcacacccaatgtatcggttaacgatcagggtcaggtgacgattacctaccaaacctatagcgaagtggctgtgacggcgaaaagtaaaaaattcccaagttattcggtgagttatcggttctacccaaatcggtggatatacgatggcggcagatcgctggtatccagtctcgaggccagcagacaatgccaaggttcagatatgtctgcggttcttgaatcctcacgtgcaaccaacggaacgcgtgcgcctgacgggacattgtggggcgagtgggggagcttgaccgcgtatagttctgattggcaatctggtgaatattgggtcaaaaagaccagcacggattttgaaaccatgaatatggacacaggcgcactgcaaccagggcctgcatacttggcgttcccgctctgtgcgctgtcaatacgccgcgcgcgcagcgtggcgagcatggaagttcagctggttgaatctggtggtggtctggttcaggcgggtggttctctgcgtctgtcttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttcaccatctctcgtgacaacgcgaaaaacaccgtttacctgcagatgaactctctgaaaccggaagacaccgcggtttactactgcaaacgtttccgtaccgcggcgcagggtaccgactactggggtcagggtacccaggttaccgtttcttctcaccaccaccaccaccactaatag

SEQ ID NO: 42 - DNA, 375 bp, synthetic construct
  source 1..375: mol_type = other DNA
atggaagttcagctggttgaatctggtggtggtctggttcaggcgggtggttctctgcgtctgtcttgcgcggcgtctggtatcaccttctctatcaacaccatgggttggtaccgtcaggcgccgggtaaacagcgtgaactggttgcgctgatctcttctatcggtgacacctactacgcggactctgttaaaggtcgtttcaccatctctcgtgacaacgcgaaaaacaccgtttacctgcagatgaactctctgaaaccggaagacaccgcggtttactactgcaaacgtttccgtaccgcggcgcagggtaccgactactggggtcagggtacccaggttaccgtttcttctcaccaccaccaccaccac